(19) United States
(12) Patent Application Publication
(10) Pub. No.: US 2024/0359151 A1
(43) Pub. Date: Oct. 31, 2024

(54) ELECTROSTATIC SPRAY DRIED OIL POWDERS AND PRODUCTION METHOD THEREOF

(71) Applicant: SPRAYING SYSTEMS CO., Wheaton, IL (US)

(72) Inventors: Bogdan Zisu, Truganina (AU); AKM MASUM, Truganina (AU); Bao Loc PHAM, Truganina (AU); Michel THENIN, Aurora, IL (US); Audrey MAUDHUIT, Treillieres (FR); Elodie BEAUPEUX, Treillieres (FR)

(21) Appl. No.: 18/763,208

(22) Filed: Jul. 3, 2024

Related U.S. Application Data
(63) Continuation of application No. PCT/US2022/054149, filed on Dec. 28, 2022.

(51) Int. Cl. B01J 13/04; A23D 7/005; A23D 7/01; A23D 7/04; C12N 1/20

(52) U.S. Cl. CPC B01J 13/043; A23D 7/0053; A23D 7/011; A23D 7/04; C12N 1/20

(57) ABSTRACT

Provided is a method of providing an oil emulsion powder comprising electrostatic spray drying an emulsion comprising at least one oil, an encapsulating agent, and optionally an emulsifier at an inlet temperature of 150° C. or below and an exhaust temperature of 100° C. or below.

### CROSS-REFERENCE TO RELATED APPLICATION

This patent application claims the benefit of PCT International Patent Application No. PCT/US2022/054149, filed December 2022 and U.S. Provisional Patent Application No. 63/296,083, filed Jan. 3, 2022, which are incorporated by reference.

### BACKGROUND OF THE INVENTION

Encapsulated oil powders have extensive use across a variety of industries, including food, beverages, cosmetics, and nutraceuticals. The encapsulation provides a barrier against, for example, oxygen, light, and free radicals (Desai et al., Journal of Microencapsulation, 22 (2), 179-192 (2005)).

Oil powders typically are produced using a spray drying system. However, such systems require high inlet and outlet temperatures, which can risk degrading the oil or other components of the powder (Anwar et al., Journal of Food Engineering, 105, 367-378 (2011)). Thus, there remains a need to effectively provide an oil powder that is shelf stable, has improved loading capacity, and/or improved encapsulation efficiency.

### BRIEF SUMMARY OF THE INVENTION

The invention provides a method of providing an oil emulsion powder comprising electrostatic spray drying an emulsion comprising at least one oil, an encapsulating agent, and optionally an emulsifier at an inlet temperature of 150° C. or below and an exhaust temperature of 100° C. or below.

### BRIEF DESCRIPTION OF THE SEVERAL VIEWS OF THE DRAWINGS

FIG. 1 is a vertical section of an illustrated spray drying system for processing an oil-containing emulsion into powder form according to an embodiment of the invention.

FIG. 2 is an enlarged vertical section of the electrostatic spray nozzle assembly of the illustrated spray drying system.

FIG. 3 shows the scanning electron micro (SEM) images of 20%, 50%, and 80% (w/w) vegetable oil load powders encapsulated by electrostatic spray dried (ESD) and spray dried (SD) at 5,000× magnification.

FIGS. 4A and 4B are SEM images of 50% and 80% (w/w) ESD powders comprising either coconut oil and medium-chain-triglycerides (MCT) from coconut (FIG. 4A) or flaxseed oil and olive oil (FIG. 4B), each at 5,000× magnification.

FIG. 5 shows the SEM images of oil encapsulated powders containing 50% and 80% (w/w) fish oil and ghee at 5,000× magnification.

FIG. 6 shows the SEM images of oil encapsulated powders containing 50% (w/w) of either orange oil or mint oil, each at 5,000× magnification.

FIG. 7 shows the bacteria counts (log cfu/g, at 1% starter culture addition) for S. thermophilus (ST) and L. bulgaricus (LB) at day 0 and after storage at 4° C. for 90 days.

FIG. 8 shows the SEM images of encapsulated oil-bacteria powders at 10,000× magnification.

FIG. 9 shows the SEM images of 40% oil load DHA oil powders prepared by conventional spray drying (CSD) and electrostatic spray drying (ESD) with different encapsulants: (i) maltodextrin and casein, (ii) maltodextrin and methylcellulose, and (iii) maltodextrin and saponin.

While the invention is susceptible of various modifications and alternatives, certain illustrative embodiments will be described below in detail. It should be understood, however, that there is no intention to limit the invention to the specific forms disclosed, but on the contrary, the intention is to cover all modifications, alternative constructions, and equivalents falling within the spirit and scope of the invention.

### DETAILED DESCRIPTION OF THE INVENTION

The present invention is predicated, at least in part, on the surprising discovery that oil powders that are spray dried using a traditional high heat spray drying system compared to a low heat, electrostatic spray system are limited in their loading capacity and/or encapsulation efficiency. In accordance with this discovery, the invention provides a method of providing an oil emulsion powder comprising electrostatic spray drying an emulsion comprising at least one oil, an encapsulating agent, and optionally an emulsifier at an inlet temperature of 150° C. or below and an exhaust temperature of 100° C. or below. The produced oil powder has at least one benefit over a corresponding oil powder produce by spray drying, such as being more shelf stable, improved loading capacity, and/or improved encapsulation efficiency, compared to a comparable oil powder prepared using spray drying.

In the method, the inlet temperature is any suitable temperature that provides an oil powder with the features described herein. Typically, the inlet temperature is 150° C. or below (e.g., about 140° C. or below, about 135° C. or below, about 130° C. or below, about 125° C. or below, about 120° C. or below, about 115° C. or below, about 110° C. or below, about 105° C. or below, about 100° C. or below, about 95° C. or below, or about 90° C. or below). In some embodiments, the inlet temperature is about 140° C. or below, about 100° C. or below, about 150° C., about 140° C., or about 90° C. In comparison, conventional spray drying systems have a much higher inlet temperature, typically about 140° C. or higher, e.g., 180-250° C.

In the method, the outlet temperature is any suitable temperature that provides an oil powder with the features described herein. Typically, the outlet temperature is about 80° C. or below (e.g., about 75° C. or below, about 70° C. or below, about 65° C. or below, about 60° C. or below, about 55° C. or below, about 50° C. or below, about 45° C. or below, about 40° C. or below, about 35° C. or below). In some embodiments, the outlet temperature is about 60° C. or below, about 50° C. or below, about 60° C., about 50° C., or about 35° C. In comparison, conventional spray drying systems have outlet temperatures that are above 60° C., typically about 95° C.

The atomizing temperature of the electrostatic spray drying system also is relatively low, such as about 100° C. or below (e.g., about 95° C. or below, about 90° C. or below, about 85° C. or below, about 80° C. or below, about 75° C. or below, about 70° C. or below, about 65° C. or below, about 60° C. or below, about 55° C. or below, about 50° C. or below, about 45° C. or below, about 40° C. or below, about 35° C. or below, or about 30° C. or below).

The electrostatic spray drying process applies a voltage to the spray droplets, which typically is about 0.1 kV or more (e.g., about 0.5 kV or more, about 1 kV or more, about 2 kV or more, about 4 kV or more, about 5 kV or more, about 7 kV or more, about 9 kV or more, about 12 kV or more, or about 15 kV or more). The upper limit of the applied voltage typically is 30 kV and in some instances, the upper limit is 20 kV or more preferably 15 kV. Any two of the foregoing endpoints can be used to define a close-ended range, or a single endpoint can be used to define an open-ended range. In the drying process, the applied voltage can be either continuous or modulated between two or more different voltages, known as Pulsed Width Modulation (PWM). Any two or more applied voltages ranging between 0.1-30 kV (e.g., 0.5 kV and 1 kV, 1 kV and 5 kV, 1 kV and 10 kV, 5 kV and 15 kV) can be used for PWM to provide a desired effect, such as a particular agglomerate size. In some embodiments of the method, the applied voltage is continuous. In other embodiments of the method, the applied voltage is modulated between two or more different voltages, e.g., alternating between 1 kV and 10 kV.

Alternatively, or in addition, to PWM, the charge (positive or negative) of the applied voltage can be altered, as necessary. Without wish to be bound by any theory, it is believed that alternating the electrostatic charge can change the surface composition of the particle and/or the agglomeration properties. For example, an applied negative charge will allow more conductive compounds to move towards the surface of the particle and non-conductive compounds will remain near the core of the particle. Accordingly, a negative electrostatic charge typically is applied in the electrostatic spray dry process. In some embodiments, alternating the charge of the applied voltage is used when preparing an oil powder.

The oil to be used in the method is any suitable oil can be subjected to the electrostatic spray dry process. In some aspects, the at least one oil is plant or animal in origin. In some aspects, the oil is an edible oil. The oil can be provided by any source, including purchased commercially or extracted from a suitable plant (including a leaf, stem, root, nut, or seed) or animal source. Extraction can be by any suitable method, such as chemical solvent extraction and/or pressing.

Examples of the oil include vegetable oil, vegetable shortening, castor oil, rice brain oil, olive oil, canola oil, corn oil, palm oil, coconut oil, flaxseed oil, hempseed oil, rapeseed oil, linseed oil, grapeseed oil, rosehip seed oil, pomegranate seed oil, watermelon seed oil, seabuckthorn berry oil, camellia seed oil (tea oil), cranberry seed oil, hemp seed oil, borage seed oil, evening primrose oil, argan oil, jojoba oil, marula oil, carrot oil, sesame seed oil, sunflower oil, shea nut oil, soybean oil, peanut oil, walnut oil, almond oil, hazelnut oil, kukui nut oil, pecan oil, macadamia nut oil, meadowfoam oil, avocado oil, apricot kernel oil, an essential oil, silicone oil, fish oil, cocoa butter, shea butter, butter, ghee, medium chain triglycercides (MCT), and any combination thereof. Examples of an essential oil include, for example, aniseed oil, basil oil, bay oil, bergamot oil, cinnamon oil, clove oil, lavender oil, eucalyptus oil, lavender oil, ginger oil, geranium oil, rose oil, blue tansy oil, tea tree oil, moringa oil, lemon balm essential oil, lemongrass oil, thyme oil, rosemary oil, mint oil, lemon oil, orange oil, grapefruit oil, and fennel oil.

The encapsulating agent is any agent capable of encapsulating the oil. In a preferred aspect of the invention, the encapsulating agent is a carbohydrate, a lipid, a protein, ascorbic acid, or a combination thereof.

In an aspect, the carbohydrate can be, e.g., maltodextrin, sucrose, dextrose, glucose, lactose, trehalose, amylase, cyclodextrin, dextrin, galactomannan, pectin, starch (e.g., corn starch, waxy maize starch, native tapioca starch, pea starch), modified food starch (e.g., modified tapioca starch, OSA (octenyl succinic anhydride) modified starch), inulin, gum Arabic, guar gum, gellan gum, mesquite gum, xanthan gum, alginate, chitosan, shellac, carboxymethylcellulose, or a combination thereof. Maltodextrins are usually classified by their dextrose equivalent value (DE) that range from 1 to 20. Maltodextrins with DE values of 4, 6, 10, 12, 15, 19, 20, 25, 30, and 42 are commercially available. Sources of maltodextrin include, e.g., maize, tapioca, and rice.

In an aspect, the lipid can be, e.g., a fatty acid or an ester thereof, a fatty alcohol or an ester thereof, a triglyceride, a phospholipid, a glycolipid, an aminolipid, a lipopeptide, partial acylglycerol, or a combination thereof. Examples of a suitable lipid include, e.g., carnauba wax, candelilla wax, beeswax, solid paraffin, rice bran wax, hydrogenated soybean oil, hydrogenated palm oil, palmitic acid, stearic acid, behenic acid, lauric acid, glyceryl tripalmitate glyceryl trimyristate, glyceryl trilaurate, cetyl alcohol, lauryl alcohol, stearyl alcohol, oleyl alcohol, and lecithin.

In an aspect, the protein can be protein from a plant source or an animal source (e.g., milk). Examples of proteins include, e.g., casein, a caseinate (e.g., sodium caseinate, calcium caseinate, calcium phosphate caseinate), gelatin, casein, soy protein, wheat protein, whey protein, rice protein, pea protein, cocoa shell protein, or a combination thereof.

In an aspect of the method, the processing conditions provide an emulsion between the at least one oil and encapsulating agent. In another aspect, the emulsion comprises an emulsifier. The emulsifier is any suitable surfactant that enables the production of an emulsified powder between the oil and the encapsulating agent. One or more than one (e.g., 2, 3, 4, etc.) emulsifiers can be used in the composition. In some aspects, the emulsifier is at least one selected from casein, a caseinate (e.g., sodium caseinate, calcium caseinate, calcium phosphate caseinate), lecithin, saponin (e.g., quilaja, glycyrrhizic acid), carrageenan, gum Arabic (GA), xanthan, whey protein isolate (WPI), whey protein concentrate (WPC), stearate, glyceryl monostearate, sucrose ester, monopropylene glycol, propylene glycol ester of fatty acid, polyglycerol esters of fatty acid, a mono- and diglycerol, mono- and diglycerides of fatty acids (e.g., citric acid ester of monoglyceride (CAEM), saturated distilled monoglyceride (SDM), polyglycerol fatty acid ester (PGFE), succinylated monoglyceride (SMG), lecithin (LC)), distilled monoglyceride, polyglycerol polyricinoleate, polysorbate 80, a sorbitan ester (e.g., polyoxyethylene (20) sorbitan monooleate, polyoxyethylene (20) sorbitan monostearate, polyoxyethylene (20) sorbitan monopalmitate, polyoxyethylene (20) sorbitan monolaurate, sorbitan monooleate, sorbitan tristearate, sorbitan monopalmitate), a lactylated ester (e.g., stearoyl lactylate), an ethoxylated ester, a succinated ester (e.g., sodium starch octenyl succinate), a fruit acid ester, carboxymethyl cellulose, and a combination thereof. In a preferred embodiment, the emulsion comprises at least one emulsifier.

In an aspect of the method, the emulsion can further comprises at least one additive that is part of the oil powder product. One additive or more than one additive (e.g., 2, 3, 4, 5, etc.) can be used. Typical additives include, e.g., an antioxidant, a vitamin, a bacterium, an omega oil, an essential oil, a flavoring agent, a pigment, a dye, and a combination thereof.

An antioxidant can be used to inhibit oxidation and help stabilize the oil. Suitable antioxidants include, for example, butylated hydroxyanisole (BHA), butylated hydroxytoluene (BHT), propyl gallate (PG), tert-butyl hydroquinone (TBHQ), β-carotene, ascorbic acid, tocopherol, tea extract, rosemary extract, sage extract, thyme extract, alkannin, shikonin, ascorbyl palmitate, and a flavonoid (e.g., catechin, apicatechins, epicatechin gallate, epigallocatechin, and epigallocatechin gallate).

Vitamins include, for example, vitamin A, B, C, D, E, and K, including the vitamers of each thereof.

The bacterium includes, for example, a starter culture, a probiotic, and a combination thereof. The starter culture can be, for example, from the genus lactobacillus, streptococcus, and leuconostoc. Specific examples of a starter culture include, e.g., L. bulgaricus, L. lactis, L. acidophilus, L. helveticus, L. casei, L. plantarum, L. rhamnosus, Leuconostoc citrovorum, Leuconostoc dextranicum, S. lactis, S. cremoris, S. diacetylactis, S. durans, S. faecalis, S. thermophilus, propionic bacterium shermanii, and combinations thereof. Suitable probiotics include those from the genus bifidobacteria, lactobacillus, and saccharomyces, preferably bifidobacteria or lactobacillus. Specific examples of probiotic include, e.g., B. animalis, B. breve, B. lactis, B. longum, L. acidophilus, L. reuteri, S. bourladii, and combinations thereof.

The omega oil, also known as an omega-3 oil, is a polyunsaturated fatty acid with a double bond three atoms away from the terminal methyl group. Examples include, eicosapentaenoic acid (EPA), docosahexaenoic acid (DHA), α-linolenic acid (ALA), and a combination thereof.

The essential oil as an additive is as described herein.

The flavoring agent can be in the form of an oil, a non-aqueous solution, or an emulsion. The flavoring agent can be natural or synthetic. Suitable examples include, e.g., limonene, fenchone, vanillin, thymol, menthol, isoamyl acetate, benzaldehyde, ethyl propionate, ethyl butyrate, methyl anthranilate, methyl salicylate, ethyl decadienoate, allyl hexanoate, ethyl maltol, 2,4-dithiapentance, fumaric acid, acetic acid, ascorbic acid, citric acid, lactic acid, malic acid, phosphoric acid, tartaric acid, citral, massoia lactone, acetoin, manzanate, cinnamaldehyde, a glutamate (e.g., mono- and/or disodium glutamate), a glycine salt, a guanylic acid salt, an inosinic acid salt, and a 5′-ribonucleotide salt.

The pigment or dye can be natural or synthetic and include, for example, a mineral, a clay, charcoal, carbon black, ultramarine, ultramarine green shade, Tyrian red, Indian yellow, a cadmium-based pigment (e.g., cadmium yellow, cadmium ted, cadmium green, cadmium orange), a chromium-based pigment (e.g., chrome yellow, chrome green), a cobalt-based pigment (e.g., cobalt violet, cobalt blue, cerulean blue, aureolin), a copper-based pigment (e.g., azurite, Han purple, Han blue, an Egyptian blue, malachite, Paris green, phthalocyanine blue BN, phthalocyanine Green G, verdigris), iron oxide-based pigment (e.g., sanguine, caput mortuum, oxide red, red ochre, yellow ochre, Venetian red, Prussian blue), a lead-based pigment (e.g., white lead, red lead, cremnitz white, Naples yellow), a manganese-based pigment (e.g., manganese violet, YInMn blue), a titanium-based pigment (e.g., titanium yellow, titanium beige, titanium white, titanium black), a zinc-based pigment (e.g., zinc white, zinc ferrite, zinc yellow), a marine based pigment (e.g., chlorophyll a, b, and c, β-carotene, phycocyanin, xanthophyll, phycoerythrin), aluminum powder, vermillion, an aniline dye (e.g., mauveine, aniline yellow), an azo dye (e.g., C.I. Direct Black 171, sunset yellow, tartrazine, azorubine, ponceau, amaranth, allura red), an acid dye (e.g., Indian ink, congo red, nigrosoine), a naphthol (an azoic) dye, a nitro dye (e.g., maritus yellow), an anthraquinone dye (e.g., C.I. Reactive Blue 19, indanthrone, alizarin, 1-aminoanthraquinone), a sulfur dye (e.g., indophenol, sulfur black, sulfur red 7), turmeric, and combinations thereof.

In an aspect of the invention, the oil emulsion powder produced by the method has a lower amount of surface free fat compared to a spray dried powder of the same oil emulsion. For example, the oil powder product can have 40% surface free fat or less (e.g., 37% or less, 35% or less, 30% or less, 25% or less, 20% or less, 10% or less, 8% or less, 6% or less, 5% or less, 4% or less, 3% or less, 2% or less, 1% or less, 0.5% or less, or 0.2% or less).

In an aspect of the invention, the oil emulsion powder has any suitable oil load (e.g., about 1-90%). For example, the oil load can be about 1% or more (e.g., about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, or about 85% or more). The upper limit of the oil load is typically about 90% or less (e.g., about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, or about 10% or less). Any two of the foregoing endpoints can be used to define a close-ended range, or a single endpoint can be used to define an open-ended range. In an aspect, the oil powder produced by the claimed method has a higher oil load (e.g., 20% or more, 50% or more, 60% or more, 70% or more, or 80% or more, about 90%).

In an aspect of the invention, the oil emulsion powder has an encapsulation efficiency of 50% or more (e.g., 55% or more, 60% or more, 65% or more, 70% or more, 75% or more, 80% or more, 85% or more, 90% or more, 92% or more, 95% or more, 96% or more, 97% or more, 98% or more, and 99% or more).

In an aspect of the invention, the method provides an improved oil load and encapsulation efficiency compared to a spray dried powder of the same oil emulsion. In particular, the oil load of the produced oil powder can range from 1-60% in combination with an encapsulation efficiency that ranges from 90-99%. In another aspect of the invention, the oil load can range from 61-90% in combination with an encapsulation efficiency that ranges from 55-90%.

The oil powder product has a low moisture content, typically about 5% or less (e.g., about 4.5% or less, about 4% or less, about 3.5% or less, about 3% or less, about 2.5% or less, about 2% or less), preferably in combination with a low water activity (e.g., about 0.3 or less, including about 0.28 or less, about 0.25 or less, about 0.2 or less, about 0.18 or less, about 0.15 or less, or about 0.1 or less). In an aspect of the method, the oil powder product has a moisture content of about 4% or less or about 3% or less.

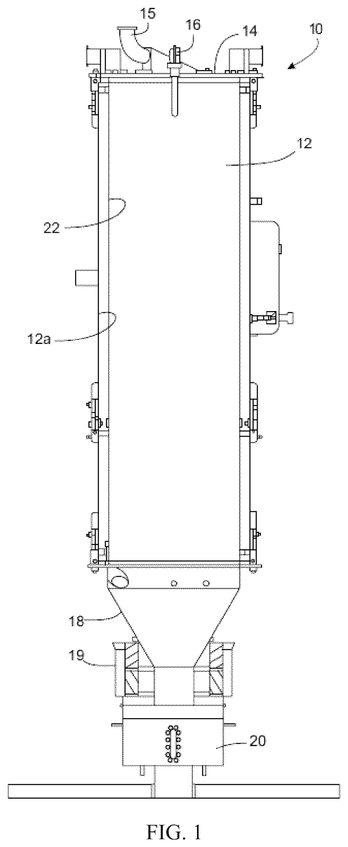

Referring now more clearly to the drawings, FIG. 1 is an illustrated spray drying system 10 for processing an oil-containing emulsion into powder form according to the invention. A basic construction and operation of the illustrated spray drying system 10 is similar to that disclosed in U.S. Pat. No. 10,286,411, assigned to the same assignee as the present application, the disclosure of which is incorporated herein by reference.

The spray drying system 10 in this case includes a processing tower 11 comprising a drying chamber 12 in the form of an upstanding cylindrical structure, a top closure arrangement in the form of a cover or lid 14 for the drying chamber 12 having a heating air inlet 15 and a liquid spray nozzle assembly 16, and a bottom closure arrangement in the form of a powder collection cone 18 supported at the bottom of the drying chamber 12, a filter element housing 19 through which the powder collection cone 18 extends having a heating air exhaust outlet, and a bottom powder collection chamber 21.

The illustrated drying chamber 12 has a “replaceable internal non-metallic” insulating liner 22 disposed in concentric spaced relation to the inside wall surface 12a of the drying chamber 12 into which electrostatically charged liquid spray particles from the spray nozzle assembly 16 are discharged. The liner 100 has a diameter d less than the internal diameter d1 of the drying chamber 12 so as to provide an insulating air spacing 101 with the inner wall surface 12a of the drying chamber 12. The liner 100 preferably is non-structural being made of a non-permeable flexible plastic material.

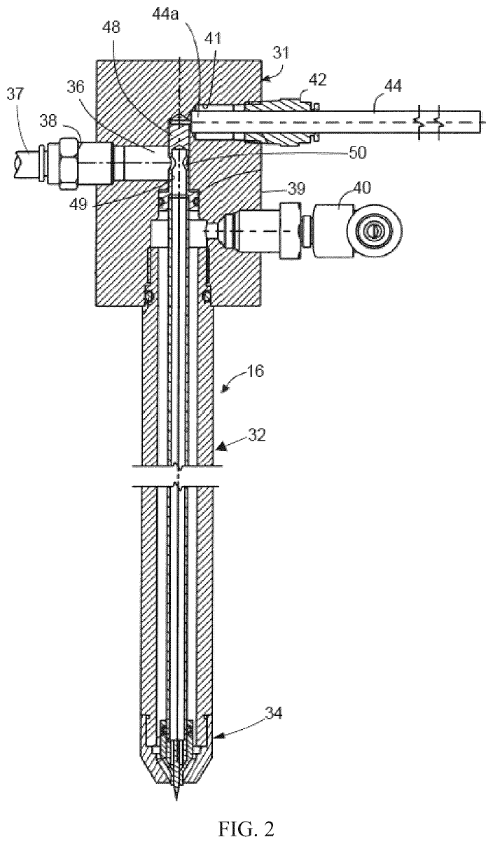

The spray nozzle assembly 16, as best depicted in FIG. 2, is a pressurized air assisted electrostatic spray nozzle assembly for directing a spray of electrostatically charged particles into the dryer chamber 12 for quick and efficient drying of an oil-containing emulsion into powder form. The illustrated spray nozzle assembly 16, includes a nozzle supporting head 31, an elongated nozzle barrel or body 32 extending downstream from the head 31, and a discharge spray tip assembly 34 at a downstream end of the elongated nozzle body 32. The head 31 in this case is made of plastic or other non-conductive material and formed with a radial liquid inlet passage 36 that receives and communicates with a liquid inlet fitting 38 for coupling to a supply line 37 that communicates with a supply of an oil powder product to be spray dried.

The nozzle supporting head 31 in this case further is formed with a radial pressurized air atomizing inlet passage 39 downstream of said liquid inlet passage 36 that receives and communicates with an air inlet fitting 40 coupled to a suitable pressurized gas supply. The head 31 also has a radial passage 41 upstream of the liquid inlet passage 36 that receives a fitting 42 for securing a high voltage cable 44 connected to a high voltage source and having an end 44a extending into the passage 41 in abutting electrically contacting relation to an electrode 48 axially supported within the head 31 and extending downstream of the liquid inlet passage 36.

For enabling liquid passage through the head 31, the electrode 48 is formed with an internal axial passage 49 communicating with the liquid inlet passage 36 and extending downstream though the electrode 48. The electrode 48 is formed with a plurality of radial passages 50 communicating between the liquid inlet passage 36 and the internal axial passage 49.

The elongated body 32 is in the form of an outer cylindrical body member 55 made of plastic or other suitable nonconductive material, having an upstream end 55a threadably engaged within a threaded bore of the head 31. The liquid feed tube 58 is disposed in electrical contacting relation with the electrode 48 for efficiently electrically charging liquid throughout its passage from the head 31 and through elongated nozzle body member 32 to the discharge spray tip assembly 34, which in this case is similar to that disclosed in U.S. Pat. No. 10,286,411.

As will become apparent, the electrostatic spray drying system 10 is operable for drying oil powders into fine particles with improved characteristics over the prior art.

As used herein the term “about” typically refers to ±1% of a value, ±5% of a value, or ±10% of a value.

The invention is further illustrated by the following features.

(1) A method of providing an oil emulsion powder comprising electrostatic spray drying an emulsion comprising at least one oil, an encapsulating agent, and optionally an emulsifier at an inlet temperature of 150° C. or below and an exhaust temperature of 100° C. or below.

(2) The method of feature 1, wherein the atomizing temperature is about 100° C. or below.

(3) The method of feature 1 or 2, wherein the applied voltage is about 0.1 kV or more.

(4) The method of any one of features 1-3, wherein the applied voltage is continuous.

(5) The method of any one of features 1-4, wherein the applied voltage is modulated between two or more different voltages.

(6) The method of any one of features 1-4, wherein the applied voltage alternates charges.

(7) The method of any one of features 1-6, wherein the oil emulsion powder has lower amount of surface free fat compared to a spray dried powder of the same oil emulsion.

(8) The method of any one of features 1-7, wherein the oil emulsion powder has an encapsulation efficiency of 50% or more.

(9) The method of feature 8, wherein the oil load ranges from 1-60% and the encapsulation efficiency ranges from 90-99%.

(10) The method of feature 8, wherein the oil load ranges from 61-90% and the encapsulation efficiency ranges from 55-90%.

(11) The method of any one of features 1-10, wherein the at least one oil is plant or animal in origin.

(12) The method of feature 11, wherein the at least one oil is vegetable oil, vegetable shortening, castor oil, rice brain oil, olive oil, canola oil, corn oil, palm oil, coconut oil, flaxseed oil, hempseed oil, rapeseed oil, linseed oil, grapeseed oil, rosehip seed oil, pomegranate seed oil, watermelon seed oil, seabuckthorn berry oil, camellia seed oil (tea oil), cranberry seed oil, hemp seed oil, borage seed oil, evening primrose oil, argan oil, jojoba oil, marula oil, carrot oil, sesame seed oil, sunflower oil, shea nut oil, soybean oil, peanut oil, walnut oil, almond oil, hazelnut oil, kukui nut oil, pecan oil, macadamia nut oil, meadowfoam oil, avocado oil, apricot kernel oil, an essential oil, silicone oil, fish oil, cocoa butter, shea butter, butter, ghee, medium chain triglycercides (MCT), or any combination thereof.

(13) The method of any one of features 1-12, wherein the encapsulating agent is a carbohydrate, a lipid, a protein, ascorbic acid, or a combination thereof.

(14) The method of feature 13, wherein the carbohydrate is maltodextrin, sucrose, dextrose, glucose, lactose, trehalose, amylase, cyclodextrin, dextrin, galactomannan, pectin, starch, modified food starch, inulin, gum Arabic, guar gum, gellan gum, mesquite gum, xanthan gum, alginate, chitosan, shellac, carboxymethylcellulose, or a combination thereof.

(15) The method of feature of 13 or 14, wherein the lipid is a fatty acid or an ester thereof, a fatty alcohol or an ester thereof, a triglyceride, a phospholipid, a glycolipid, an aminolipid, a lipopeptide, partial acylglycerol, or a combination thereof.

(16) The method of any one of features 13-15, wherein the protein is casein, caseinate, gelatin, soy protein, wheat protein, whey protein, rice protein, pea protein, cocoa shell protein, or a combination thereof.

(17) The method of any one of features 1-16, wherein the emulsion comprises an emulsifier.

(18) The method of features 17, wherein the emulsifier is at least one selected from casein, caseinate, lecithin, saponin, carrageenan, gum Arabic, xanthan, whey protein isolate, stearate, glyceryl monostearate, sucrose ester, monopropylene glycol, propylene glycol ester of fatty acid, polyglycerol esters of fatty acid, a mono- and diglycerol, mono- and diglycerides of fatty acids, distilled monoglyceride, polyglycerol polyricinoleate, polysorbate 80, a sorbitan ester, a lactylated ester, an ethoxylated ester, a succinated ester, a fruit acid ester, carboxymethyl cellulose, and a combination thereof.

(19) The method of any one of features 1-18, wherein the emulsion further comprises at least one additive.

(20) The method of feature 19, wherein the at least one additive is an antioxidant, a vitamin, a bacterium, an omega oil, an essential oil, a flavoring agent, a pigment, a dye, or a combination thereof.

The following examples further illustrate the invention but, of course, should not be construed as in any way limiting its scope.

### Example 1

This example demonstrates low temperature electrostatic spray drying of an oil powder product in an embodiment of the invention.

Oil emulsion powders were made by electrostatic spray drying (ESD) at inlet temperatures of 90° C., 140° C., and 150° C., however, the inlet drying temperature can be as low as 80° C. Atomizing temperature was generally maintained below 80° C. and the exhaust temperature below 60° C. In this example, the atomizing temperature was set to 35° C., 50° C., and 80° C. to obtain exhaust temperatures of 35° C., 50° C., and 60° C., respectively. Negative pulsed width modulation (PWM) alternating between 10 kV and 1 kV was used in these examples, however, electrostatic charge may be positive, and it may be as low as 0.1 kV and as high as 20 kV with or without PWM. Atomizing gas pressure may range from 30-552 kPa. For comparison, oil emulsion powders were also spray dried at 180° C. inlet and 90° C. exhaust by conventional high heat spray drying. The processing parameters are shown in Table 1.

TABLE 1

| | | | | Spray |
|---|---|---|---|---|
| | ESD | ESD | ESD | Dried |
| | (90/ | (140/ | (150/ | (180/ |
| Parameter | 35° C.) | 50° C.) | 60° C.) | 95° C.) |
| Inlet temp (° C.) | 90 | 140 | 150 | 180 |
| Outlet temp (° C.) | 35 | 50 | 60 | 95 |
| Atomizing gas pressure (kPa) | 210 | 210 | 210 | 100 |
| PWM voltage (High/Low) (kV) | 10/1 | 10/1 | 10/1 | NA |
| Charge | −ve | −ve | −ve | NA |

ESD = electrostatic spray dried

### Example 2

This example demonstrates low temperature electrostatic spray drying of a vegetable oil powder product in an embodiment of the invention.

Vegetable oil emulsions were formulated to contain 20% to 90% (w/w) vegetable oil, encapsulated with maltodextrin and stabilized with sodium caseinate. Oil emulsions were spray dried (SD) at 180° C. and electrostatic spray dried (ESD) with 10 kV PWM at 90° C. and 140° C. Table 2 shows the moisture content and water activity of the resulting vegetable oil powders. Moisture content was below 3% in all powders and water activity below 0.22.

TABLE 2

| | Moisture content | Water |
|---|---|---|
| | solids-non-fat | activity (a_{w}) |
| Powder | (% w/w ± sd) | (a_{w} ± sd) |
| ESD20% Oil_90/35 | 2.74 ± 0.72 | 0.1573 ± 0.0213 |
| ESD20% Oil_140/50 | 1.29 ± 0.02 | 0.0843 ± 0.0053 |
| ESD50% Oil_90/35 | 2.28 ± 0.51 | 0.1657 ± 0.0040 |
| ESD50% Oil_140/50 | 1.18 ± 0.06 | 0.0937 ± 0.0072 |
| ESD60% Oil_140/50 | 1.56 ± 0.44 | 0.1508 ± 0.0112 |
| ESD70% Oil_140/50 | 1.18 ± 0.16 | 0.1556 ± 0.0107 |
| ESD80% Oil_140/50 | 1.03 ± 0.18 | 0.1882 ± 0.0324 |
| ESD90% Oil_140/50 | 1.45 ± 0.07 | 0.2194 ± 0.0085 |
| SD20% Oil_180/95 | 0.51 ± 0.09 | 0.0969 ± 0.0021 |
| SD50% Oil_180/95 | 0.65 ± 0.10 | 0.1116 ± 0.0073 |
| SD80% Oil_180/95 | 1.03 ± 0.25 | 0.1924 ± 0.0085 |

sd = standard deviation
ESD = electrostatic spray dried
SD = spray dried
90/35, 140/50, 180/95 = inlet and outlet drying temperatures

Table 3 shows the oil load, surface free fat, encapsulation efficiency, and peroxide value of vegetable oil powders. The peroxide values (in all of the examples except Example 7) were measured soon after preparation of the powders using the spectrophotometric standard method of the International Dairy Federation (IDF) (see, e.g., Rahmani-Manglano et al., Foods, 9, 545, 21 pages (2020)). Alternative methods of measuring the peroxide value include, e.g., the titration method of the International Fragrance Association (IFRA) and American Oil Chemists' Society (AOCS) Official Method Cd 8b-90 (see also, e.g., Selim et al., Molecules, 26, 6109, 17 pages (2021) and Shantha et al., Journal of AOAC International, 77 (2), 421-424 (1994)).

Electrostatic spray drying produced powders with greater encapsulation efficiency than spray drying at 20%, 50%, and 80% oil load. Overall, the encapsulation efficiency drops with increasing oil load. At 20% oil load, the encapsulation efficiency was greater than 99% in ESD powders and lower than 97% in spray dried powders. At 50% oil load, the encapsulation efficiency was more than 97% in ESD powders compared to less than 90% in spray dried powders. At 80% oil load, ESD powders had 73% encapsulation efficiency compared to 53% by spray drying.

Traditional high heat spray drying has been used to produce oil emulsion powders with up to 67% oil load with significantly lower encapsulation efficiency than the present inventive ESD method. See, e.g., Alpizar-Reyes et al., International Journal of Biological Macromolecules, 2020, 145, 207-215; Benito-Román et al., Heliyon, 2020, 6 (4), e03615-e03615; da Silva James et al., Brazilian Journal of Development, 2019, 5 (7), 8082-95; and Domian et al., Journal of Food Engineering, 2014, 125 (1), 34-43. Comparable encapsulation efficiency was reported in powders with lower oil loads of up to 22% (Benito-Román et al., 2020).

Without being bound by any theory, it is believed that the difference in encapsulation efficiency is due to the difference in surface free fat. Surface free fat is approximately three times lower in ESD powders at 20% and 50% oil load (<0.2% vs 0.6% at 20% oil; <1.5% vs 5% at 50% oil load). At 80% oil load the difference between ESD and SD is almost double (21.53% ESD vs 37.41% SD).

The peroxide value was low (below 1.8 meq O₂/kg oil) in all the powders.

TABLE 3

| | | Surface | Encapsulation | Peroxide value |
|---|---|---|---|---|
| | Oil load | free fat | efficiency | (meq O₂/kg |
| Powder | (% ± sd) | (% ± sd) | (% ± sd) | oil ± sd) |
| ESD20% Oil_90/35 | 19.94 ± 0.18 | 0.19 ± 0.06 | 99.07 ± 0.33 | 1.40 ± 0.20 |
| ESD20% Oil_140/50 | 20.42 ± 0.66 | 0.11 ± 0.03 | 99.46 ± 0.16 | 0.96 ± 0.09 |
| ESD50% Oil_90/35 | 49.88 ± 0.11 | 1.15 ± 0.39 | 97.71 ± 0.77 | 1.71 ± 0.45 |
| ESD50% Oil_140/50 | 50.04 ± 0.22 | 1.49 ± 0.01 | 97.03 ± 0.03 | 0.72 ± 0.16 |
| ESD60% Oil_140/50 | 59.83 ± 0.13 | 5.09 ± 0.52 | 91.50 ± 0.88 | 0.93 ± 0.19 |
| ESD70% Oil_140/50 | 69.69 ± 0.20 | 14.06 ± 0.35 | 79.83 ± 0.44 | 1.02 ± 0.18 |
| ESD80% Oil_140/50 | 79.89 ± 0.10 | 21.53 ± 0.49 | 73.05 ± 0.59 | 0.88 ± 0.09 |
| ESD90% Oil_140/50 | 89.55 ± 0.38 | 36.92 ± 1.08 | 58.77 ± 1.38 | 0.25 ± 0.05 |
| SD20% Oil_180/95 | 19.93 ± 0.05 | 0.62 ± 0.20 | 96.89 ± 0.99 | 1.74 ± 0.18 |
| SD50% Oil_180/95 | 49.89 ± 0.25 | 5.08 ± 0.45 | 89.83 ± 0.93 | 1.22 ± 0.10 |
| SD80% Oil_180/95 | 79.65 ± 0.45 | 37.41 ± 1.80 | 53.03 ± 2.53 | 0.21 ± 0.02 |

sd = standard deviation
ESD = electrostatic spray dried
SD = spray dried
meq = milliequivalent
90/35, 140/50, 180/95 = inlet and outlet drying temperatures

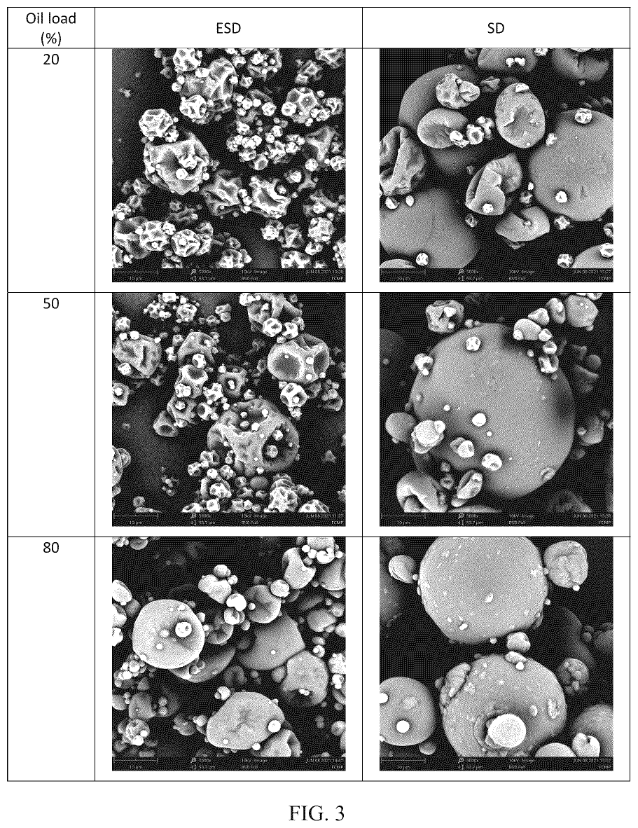

FIG. 3 shows the scanning electron micro (SEM) images of 20%, 50%, and 80% (w/w) vegetable oil load powders encapsulated by electrostatic spray dried (ESD) and spray dried (SD) at 5,000× magnification. Primary particles in SD powders were generally larger than ESD powders. At 20% and 50% oil load, there was little change in appearance, however, at 80% oil load, the physical appearance changed. In ESD powders, there was increased swelling of the primary particles, and SD powders exhibited significant particle fusion.

### Example 3

This example demonstrates low temperature electrostatic spray drying of a plant-based oil powder product in an embodiment of the invention.

Coconut oil, medium-chain-triglycerides (MCT) from coconut, flaxseed oil and olive oil emulsions were formulated to contain 50% and 80% (w/w) oil, encapsulated with maltodextrin and stabilized with sodium caseinate. Emulsions were then dried using electrostatic spray drying at temperatures of 90° C. inlet and 35° C. outlet.

Table 4 shows the moisture content and water activity of resulting powders. All the powders had a moisture content below 4% and a water activity below 0.28. Moisture and water activity were higher in powders with greater oil load.

TABLE 4

| | Moisture content solids-non-fat | Water activity (a_{w}) |
|---|---|---|
| Powder | (% w/w ± sd) | (a_{w} ± sd) |
| Coconut50% Oil | 1.44 ± 0.45 | 0.149 ± 0.029 |
| Coconut80% Oil | 3.13 ± 0.32 | 0.258 ± 0.002 |
| MCT50% Oil | 1.27 ± 0.10 | 0.179 ± 0.012 |
| MCT80% Oil | 1.80 ± 0.92 | 0.266 ± 0.022 |
| Flaxseed50% Oil | 1.46 ± 0.17 | 0.150 ± 0.007 |
| Flaxseed80% Oil | 2.13 ± 0.32 | 0.235 ± 0.017 |
| Olive50% Oil | 3.34 ± 0.57 | 0.268 ± 0.019 |
| Olive80% Oil | 3.43 ± 0.18 | 0.280 ± 0.006 |

sd = standard deviation

Table 5 shows the oil load, surface free fat, encapsulation efficiency and peroxide value of coconut oil, MCT, flaxseed oil, and olive oil powders. At 50% oil load the surface free fat was approximately 1-1.2% and this increased to 16-20% at 80% oil load in all powders. Encapsulation efficiency was >97% at 50% oil load and 74-79% in powders containing 80% oil.

The peroxide values were lowest (0.05-0.15 meq O₂/kg oil) in coconut oil powders irrespective of oil load presumably due to the high content of saturated fats (Hee et al., The Journal of Supercritical Fluids, 2017, 130, 118-124). Flaxseed and olive oils are rich in unsaturated fatty acids (Bakry et al., Comprehensive Reviews in Food Science and Food Safety, 2008, 15 (1), 143-182; Koutsopoulos et al., Meat Science, 2008, 79 (1), 188-197), and the peroxide value increased (1.19-1.68 meq O₂/kg oil).

TABLE 5

| | | Surface | Encapsulation | Peroxide value |
|---|---|---|---|---|
| | Oil load | free fat | efficiency | (meq O₂/kg |
| Powder | (% ± sd) | (% ± sd) | (% ± sd) | oil ± sd) |
| Coconut50% Oil | 50.26 ± 0.28 | 1.20 ± 0.16 | 97.61 ± 0.32 | 0.11 ± 0.04 |
| Coconut80% Oil | 80.05 ± 0.15 | 18.06 ± 0.79 | 77.44 ± 1.03 | 0.05 ± 0.01 |
| MCT50% Oil | 50.34 ± 0.23 | 1.04 ± 0.18 | 97.93 ± 0.36 | 0.15 ± 0.02 |
| MCT80% Oil | 80.09 ± 0.23 | 19.26 ± 3.51 | 75.96 ± 4.32 | 0.12 ± 0.01 |
| Flaxseed50% Oil | 49.70 ± 0.37 | 1.23 ± 0.20 | 97.53 ± 0.38 | 1.19 ± 0.15 |
| Flaxseed80% Oil | 79.87 ± 0.25 | 16.47 ± 1.05 | 79.39 ± 1.25 | 1.68 ± 0.12 |
| Olive50% Oil | 50.06 ± 0.25 | 1.21 ± 0.37 | 97.58 ± 0.75 | 1.48 ± 0.26 |
| Olive80% Oil | 80.04 ± 0.49 | 20.76 ± 1.58 | 74.06 ± 2.13 | 1.29 ± 0.23 |

sd = standard deviation
meq = milliequivalent

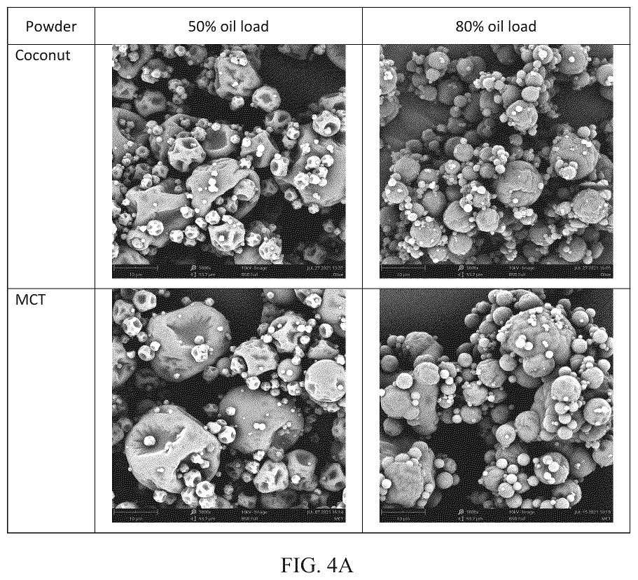

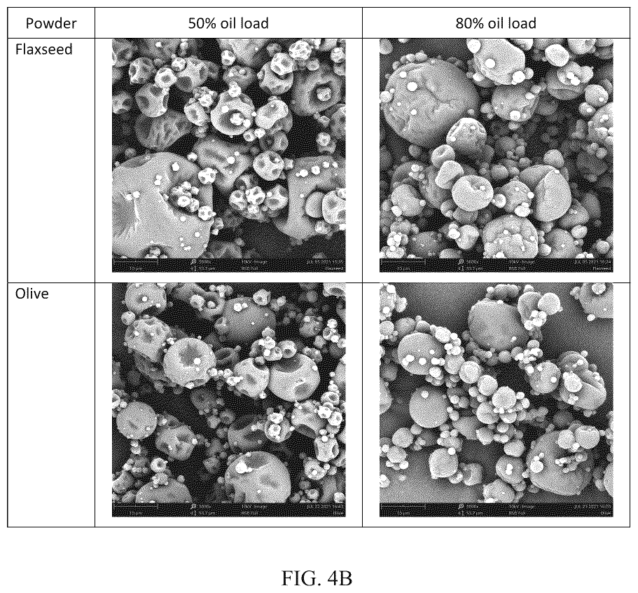

FIGS. 4A and 4B show the SEM images of 50% and 80% oil load of different oil powders. FIG. 4A shows coconut oil and MCT particles, and FIG. 4B shows flaxseed oil and olive oil particles. Primary particles were similar in appearance for all oil types at an equivalent oil load. Differences were observed between 50% and 80% oil load powders, with primary particles in the 80% oil powders showing distinct spherical appearance.

### Example 4

This example demonstrates low temperature electrostatic spray drying of an animal-based oil powder product in an embodiment of the invention.

Fish oil and ghee emulsions were formulated to contain 50% and 80% (w/w) oil, encapsulated with maltodextrin and stabilized with sodium caseinate. Emulsions were then dried using electrostatic spray drying at temperatures of 90° C. inlet and 35° C. outlet. Negative pulsed width modulation (PWM) alternating between 10 kV and 1 kV was used in these examples.

Table 6 shows the moisture content and water activity of the resulting powders. All powders had a moisture content below 3% and a water activity below 0.2 at 50% oil load and less than 0.25 at 80% oil load.

TABLE 6

| | Moisture content solids-non-fat | Water activity (a_{w}) |
|---|---|---|
| Powder | (% w/w ± sd) | (a_{w} ± sd) |
| Fish50% Oil | 1.56 ± 0.04 | 0.139 ± 0.013 |
| Fish80% Oil | 2.08 ± 0.09 | 0.231 ± 0.017 |
| Ghee50% Oil | 1.70 ± 0.23 | 0.192 ± 0.007 |
| Ghee80% Oil | 2.63 ± 0.88 | 0.244 ± 0.057 |

sd = standard deviation

Table 7 shows the oil load, surface free fat, encapsulation efficiency, and peroxide value of the fish oil and ghee powders. At 50% oil load, the surface free fat was approximately 1.1-1.3% and this increased to 17-20% at 80% oil load in all powders. The encapsulation efficiency was >97% at 50% oil load and 74-78% in powders containing 80% oil.

The peroxide values were lowest (0.14-0.31 meq O₂/kg oil) in ghee powders irrespective of oil load presumably due to the high content of saturated fats (Duhan et al., Journal of Food Processing and Preservation, 2021, 45 (6), e15537; Gupta et al., Journal of Chemical and Pharmaceutical Research, 2015, 7 (1), 568-572). Fish oils are rich in polyunsaturated fatty acids (Hashim et al., Materials Today: Proceedings, 2021, 42, 222-228; Jeyakumari et al., Journal of Food Science and Technology, 2016, 53 (1), 856-863), in this case Omega 18/12 (containing 18% eicosapentaenoic acid and 12% docosahexaenoic acid), and therefore the peroxide value increased (1.75 and 3.67 meq O₂/kg oil at 50% and 80% oil load, respectively).

TABLE 7

| | | | Encap- | Peroxide |
|---|---|---|---|---|
| | | Surface | sulation | value |
| | Oil load | free fat | efficiency | (meq O₂/kg |
| Powder | (% ± sd) | (% ± sd) | (% ± sd) | oil ± sd) |
| Fish50% Oil | 49.78 ± 0.76 | 1.29 ± 0.25 | 97.41 ± 0.54 | 1.75 ± 0.23 |
| Fish80% Oil | 79.69 ± 0.42 | 20.07 ± 2.71 | 74.83 ± 3.27 | 3.67 ± 0.16 |
| Ghee50% Oil | 50.09 ± 0.66 | 1.13 ± 0.15 | 97.75 ± 0.33 | 0.31 ± 0.05 |
| Ghee80% Oil | 80.22 ± 0.47 | 17.88 ± 2.50 | 77.70 ± 3.25 | 0.14 ± 0.02 |

sd = standard deviation
meq = milliequivalent

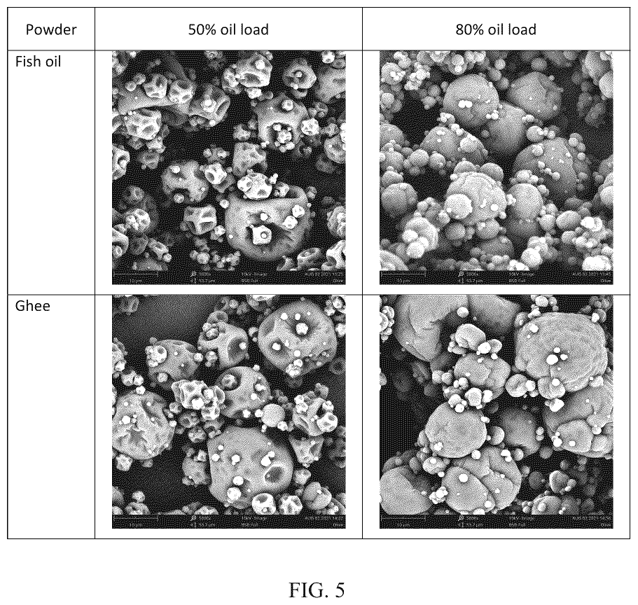

FIG. 5 shows the SEM images of 50% and 80% oil load fish oil and ghee powders. The primary particles were similar in appearance for both oil types at an equivalent oil load. Differences were observed between 50% and 80% oil load powders, with primary particles in the 80% oil powders showing a distinct spherical appearance.

### Example 5

This example demonstrates low temperature electrostatic spray drying of an essential oil powder product in an embodiment of the invention.

Orange and mint oil emulsions were formulated to contain 50% (w/w) oil, encapsulated with maltodextrin and stabilized with sodium caseinate. Emulsions were then dried using electrostatic spray drying at temperatures of 90/35° C. and 150/60° C. inlet and outlet temperatures, respectively. Negative pulsed width modulation (PWM) alternating between 10 kV and 1 kV was used in these examples

Table 8 shows the water activity of resulting powders. All powders had water activity between 0.1 and 0.23.

TABLE 8

| | Water activity (a_{w}) |
|---|---|
| Powder | (a_{w} ± sd) |
| Orange50% Oil_90/35 | 0.213 ± 0.004 |
| Orange50% Oil_150/60 | 0.100 ± 0.005 |
| Mint50% Oil_90/35 | 0.228 ± 0.003 |
| Mint50% Oil_150/60 | 0.096 ± 0.002 |

sd = standard deviation
90/35, 150/60 = inlet and outlet drying temperatures

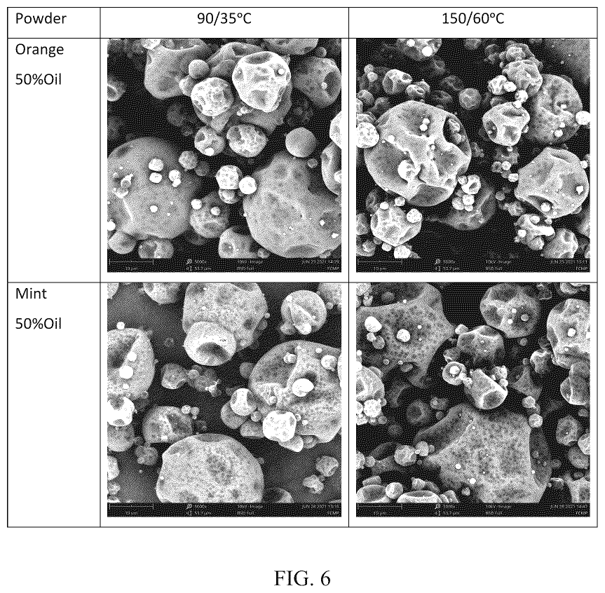

FIG. 6 shows the SEM images of 50% oil load orange and mint powders at two drying temperatures. The primary particles were similar in appearance for both oil types. Differences were observed between orange and mint oil powders, with primary particles in mint oil powders showing more distinct porous surfaces.

### Example 6

This example demonstrates low temperature electrostatic spray drying of an encapsulated oil-bacteria powder product in an embodiment of the invention.

Vegetable oil emulsions were formulated to contain 50% (w/w) oil and 1%, 10% or 20% (w/w) starter culture (S. thermophilus and L bulgaricus mixture). Maltodextrin was the encapsulant, and the emulsion was stabilized with sodium caseinate. Emulsions were dried using electrostatic spray drying at 90° C. inlet and 35° C. outlet. Negative pulsed width modulation (PWM) alternating between 10 kV and 1 kV was used in these examples

Table 9 shows the moisture content and water activity of resulting powders. All powders had a moisture content below 4% and water activity below 0.25 with 1% and 10% starter culture addition. At 20% culture addition the water activity increase to 0.3.

TABLE 9

| | Moisture content | Water |
|---|---|---|
| | solids-non-fat | activity (a_{w}) |
| Powder | (% w/w ± sd) | (a_{w} ± sd) |
| 50% Oil_1% Starter | 1.54 ± 0.21 | 0.222 ± 0.006 |
| 50% Oil_10% Starter | 2.01 ± 0.29 | 0.216 ± 0.004 |
| 50% Oil_20% Starter | 3.81 ± 0.24 | 0.299 ± 0.018 |

sd = standard deviation
Starter = starter culture

Table 10 shows the oil load, surface free fat, encapsulation efficiency, and peroxide value for oil-bacteria powders. At 1%, 10%, and 20% culture addition, the surface free fat was less than 1% and the encapsulation efficiency was >98%. The peroxide values were low (<0.2 meq O₂/kg oil).

In a similar study by Eratte et al (Journal of Functional Foods, 2015, 19, 882-892), 50% tuna oil was encapsulated with 16% (w/w) L. casei using whey protein isolate (WPI) and gum Arabic as encapsulants. Emulsions were spray dried at 180 C/80° C. (SD) and freeze-dried (FD). In Eratte et al.'s study, the surface free fat was greater (3.3% by spray drying and 11.3% by freeze drying) than recorded for electrostatic spray drying, and the encapsulation efficiency was lower (93% and 76% for SD and FD, respectively).

TABLE 10

| | | Surface | Encapsulation | Peroxide value |
|---|---|---|---|---|
| | Oil load | free fat | efficiency | (meq O₂/kg |
| Powder | (% ± sd) | (% ± sd) | (% ± sd) | oil ± sd) |
| 50% Oil_1% Starter | 50.25 ± 0.86 | 0.96 ± 0.02 | 98.10 ± 0.07 | 0.19 ± 0.03 |
| 50% Oil_10% Starter | 49.98 ± 0.37 | 0.93 ± 0.04 | 98.22 ± 0.18 | 0.16 ± 0.02 |
| 50% Oil_20% Starter | 49.52 ± 0.98 | 0.79 ± 0.06 | 98.41 ± 0.16 | 0.13 ± 0.02 |

sd = standard deviation
meq = milliequivalent
Starter = starter culture

Bacteria counts (cfu/g) for S. thermophilus (ST) and L. bulgaricus (LB) (<108) at 1-20% (w/w) starter addition are shown in Table 11. In the study by Eratte et al (2015) with 16% (w/w) L. casei addition, viability was <106 after spray drying and <108 after freeze drying. Electrostatic spray drying yields viability data were similar to freeze drying for S. thermophilus and L. bulgaricus at lower addition (1-10% here compared to 16% in Eratte et al. (2015)). At 20% addition of starter culture, the viability of S. thermophilus and L. bulgaricus were 1.82E+07 and 1.04E+09, respectively.

TABLE 11

Bacteria counts (cfu/g)

| Powder | ST | LB |
|---|---|---|
| 50% Oil_1% Starter | 7.40E+06 | 2.46E+07 |
| 50% Oil_10% Starter | 5.80E+07 | 4.80E+07 |
| 50% Oil_20% Starter | 1.82E+07 | 1.04E+09 |

ST: Streptococcus thermophilus
LB: Lactobacillus bulgaricus

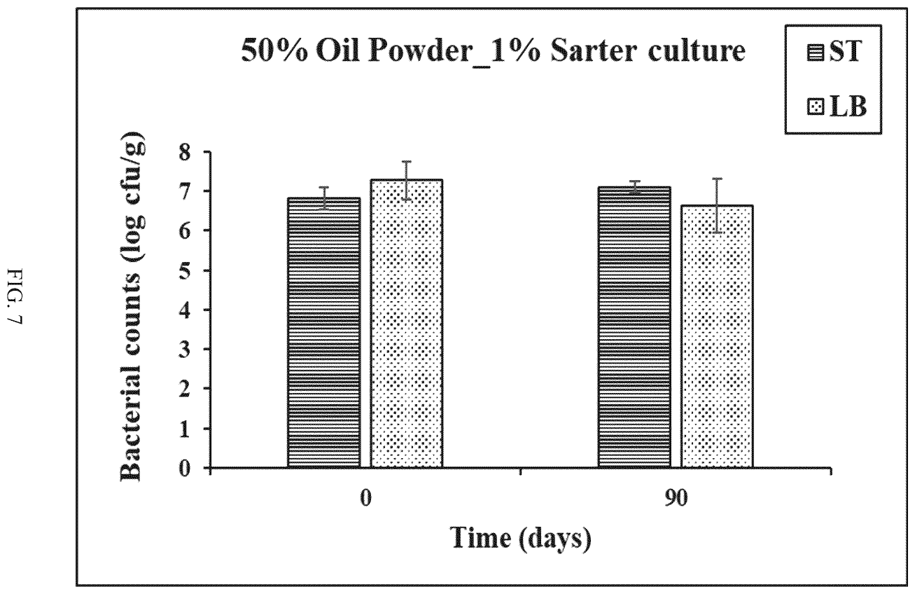

FIG. 7 shows the bacteria counts (log cfu/g, at 1% starter culture addition) for S. thermophilus (ST) and L. bulgaricus (LB) at day 0 and after storage at 4° C. for 90 days. The viability of S. thermophilus and L. bulgaricus remained higher (>7 log cfu/g for ST and >6 log cfu/g for LB) even after 90 days storage.

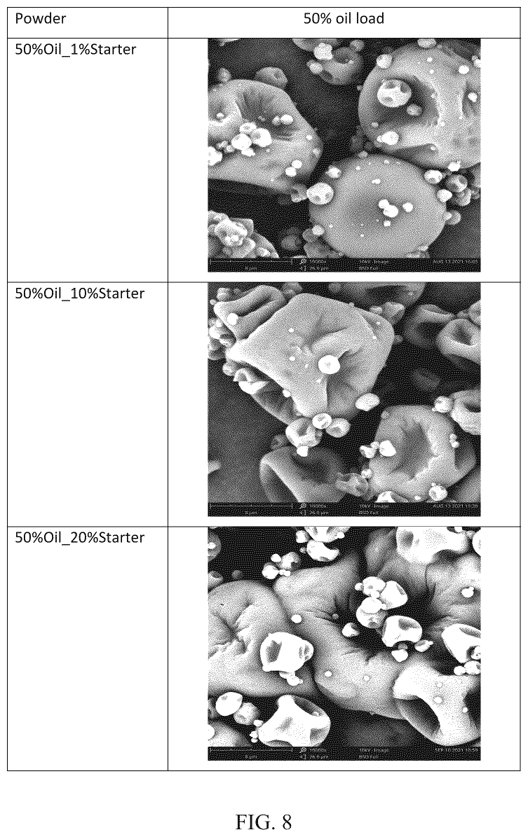

FIG. 8 shows the SEM images of the encapsulated oil-bacteria powders. The primary particles were similar in appearance irrespective of the different loads of starter bacteria.

### Example 7

This example demonstrates low temperature electrostatic spray drying of a docosahexaenoic acid (DHA) oil powder product from microalgae in an embodiment of the invention.

DHA emulsions were formulated to contain 40% (w/w) oil, encapsulated using four different formulations: (i) modified starch, (ii) maltodextrin and casein, (iii) maltodextrin and methylcellulose, and (iv) maltodextrin and saponin (Quilaja). Emulsions were dried using either conventional spray drying (CSD) at an inlet temperature of 120° C., electrostatic spray drying (ESD) at an inlet temperature of 120° C. with negative voltage at 8 kV, or freeze drying (FD).

Table 12 shows the water activity and the encapsulation efficiency. All the powders had a water activity below 0.52, but the ESD powders had a water activity below 0.23. In addition, the peroxide values were measured after 2 months of storage of the resulting powders at 40° C. in the oven using the titration method established by the International Fragrance Association (IFRA) (see, e.g., IFRA Analytical Method, “Determination of the Peroxide Value,” Sep. 10, 20219; and Kaya et al., Food Science and Technology, 141, 110872 (2021)).

TABLE 12

| | Water | Encapsulation | Peroxide Value |
|---|---|---|---|
| | activity | Efficiency | (2 months at 40° C.) |
| Powder | (a_{w}) | (% ± sd) | (meq O₂/kg oil ± sd) |
| CSD - DHA oil + Modified starch | 0.273 | 99.3 ± 0.3 | 219 ± 2 |
| CSD - Maltodextrin + casein | 0.407 | 99.3 ± 0.2 | 682 ± 5 |
| CSD - Maltodextrin + methylcellulose | 0.276 | 31.1 ± 5.9 | 1481 ± 12 |
| CSD - Maltodextrin + Saponin | 0.467 | 19.2 ± 8.0 | 232 ± 2 |
| FD - DHA oil + Modified starch | 0.436 | 98.0 ± 0.3 | 143 ± 1 |
| FD - Maltodextrin + casein | 0.390 | 99.3 ± 0.3 | 501 ± 4 |
| FD - Maltodextrin + methylcellulose | 0.512 | 34.9 ± 7.0 | 1550 ± 12 |
| FD - Maltodextrin + Saponin | 0.453 | 72.4 ± 5.4 | 351 ± 3 |
| ESD - DHA oil + Modified starch | 0.103 | 99.7 ± 0.1 | 208 ± 4 |
| ESD - Maltodextrin + casein | 0.228 | 96.1 ± 0.2 | 444 ± 2 |
| ESD - Maltodextrin + methylcellulose | 0.068 | 31.7 ± 8.2 | 365 ± 3 |
| ESD - Maltodextrin + Saponin | 0.110 | 51.9 ± 2.9 | 160 ± 1 |

sd = standard deviation
meq = milliequivalent
CSD = conventionally spray dried
FD = freeze dried
ESD = electrostatic spray dried

The encapsulation efficiency ranged between 19-100%. The formulation impacted the encapsulation efficiency.

The peroxide values were between 143 to 1550 meq O₂/kg oil after 2 months at 40° C. in the dark.

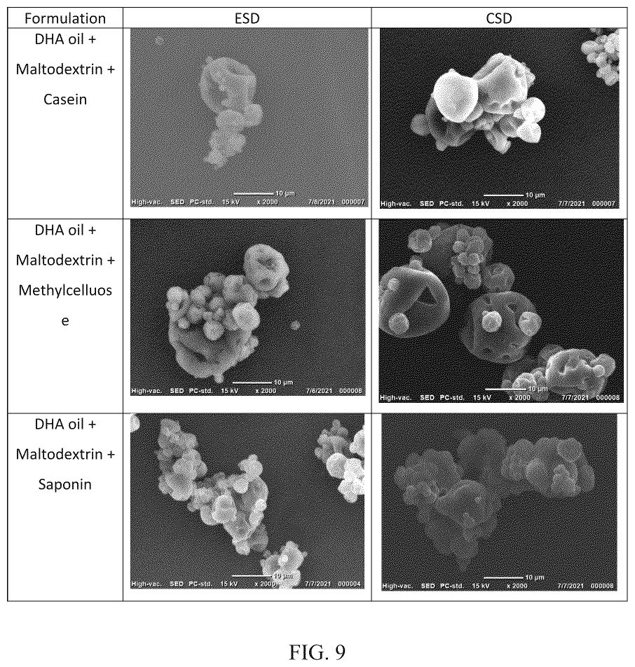

FIG. 9 shows the SEM images of 40% oil load DHA oil CSD and ESD powders with the different formulations. The primary particles were similar in appearance with the same size for all formulation. Differences were observed between the ESD and CSD powders, with a deflated balloon shape more pronounced for ESD than for CSD. The deflated balloon is characteristic of low air inlet and outlet temperatures. This shape also is likely attributed to the fact that the elastic regime is quickly reached during the drying (see, e.g., Sadek et al., Food Hydrocolloids, 48, 8-16 (2015)).

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of providing an oil emulsion powder comprising electrostatic spray drying an emulsion comprising at least one oil, an encapsulating agent, and optionally an emulsifier at an inlet temperature of 150° C. or below and an exhaust temperature of 100° C. or below.

2. The method of claim 1, wherein the atomizing temperature is about 100° C. or below.

3. The method of claim 1 or claim 2, wherein the applied voltage is about 0.1 kV or more.

4. The method of any one of claims 1-3, wherein the applied voltage is continuous.

5. The method of any one of claims 1-4, wherein the applied voltage is modulated between two or more different voltages.

6. The method of any one of claims 1-4, wherein the applied voltage alternates charges.

7. The method of any one of claims 1-6, wherein the oil emulsion powder has lower amount of surface free fat compared to a spray dried powder of the same oil emulsion.

8. The method of any one of claims 1-7, wherein the oil emulsion powder has an encapsulation efficiency of 50% or more.

9. The method of claim 8, wherein the oil load ranges from 1-60% and the encapsulation efficiency ranges from 90-99%.

10. The method of claim 8, wherein the oil load ranges from 61-90% and the encapsulation efficiency ranges from 55-90%.

11. The method of any one of claims 1-10, wherein the at least one oil is plant or animal in origin.

12. The method of claim 11, wherein the at least one oil is vegetable oil, vegetable shortening, castor oil, rice brain oil, olive oil, canola oil, corn oil, palm oil, coconut oil, flaxseed oil, hempseed oil, rapeseed oil, linseed oil, grapeseed oil, rosehip seed oil, pomegranate seed oil, watermelon seed oil, seabuckthorn berry oil, camellia seed oil (tea oil), cranberry seed oil, hemp seed oil, borage seed oil, evening primrose oil, argan oil, jojoba oil, marula oil, carrot oil, sesame seed oil, sunflower oil, shea nut oil, soybean oil, peanut oil, walnut oil, almond oil, hazelnut oil, kukui nut oil, pecan oil, macadamia nut oil, meadowfoam oil, avocado oil, apricot kernel oil, an essential oil, silicone oil, fish oil, cocoa butter, shea butter, butter, ghee, medium chain triglycercides (MCT), or any combination thereof.

13. The method of any one of claims 1-12, wherein the encapsulating agent is a carbohydrate, a lipid, a protein, ascorbic acid, or a combination thereof.

14. The method of claim 13, wherein the carbohydrate is maltodextrin, sucrose, dextrose, glucose, lactose, trehalose, amylase, cyclodextrin, dextrin, galactomannan, pectin, starch, modified food starch, inulin, gum Arabic, guar gum, gellan gum, mesquite gum, xanthan gum, alginate, chitosan, shellac, carboxymethylcellulose, or a combination thereof.

15. The method of claim of 13 or 14, wherein the lipid is a fatty acid or an ester thereof, a fatty alcohol or an ester thereof, a triglyceride, a phospholipid, a glycolipid, an aminolipid, a lipopeptide, partial acylglycerol, or a combination thereof.

16. The method of any one of claims 13-15, wherein the protein is casein, caseinate, gelatin, casein, soy protein, wheat protein, whey protein, rice protein, pea protein, cocoa shell protein, or a combination thereof.

17. The method of any one of claims 1-16, wherein the emulsion comprises an emulsifier.

18. The method of claim 17, wherein the emulsifier is at least one selected from casein, caseinate, lecithin, saponin, carrageenan, gum Arabic, xanthan, whey protein isolate, stearate, glyceryl monostearate, sucrose ester, monopropylene glycol, propylene glycol ester of fatty acid, polyglycerol esters of fatty acid, a mono- and diglycerol, mono- and diglycerides of fatty acids, distilled monoglyceride, polyglycerol polyricinoleate, polysorbate 80, a sorbitan ester, a lactylated ester, an ethoxylated ester, a succinated ester, a fruit acid ester, carboxymethyl cellulose, and a combination thereof.

19. The method of any one of claims 1-18, wherein the emulsion further comprises at least one additive.

20. The method of claim 19, wherein the at least one additive is an antioxidant, a vitamin, a bacterium, an omega oil, an essential oil, a flavoring agent, a pigment, a dye, or a combination thereof.
